(19) United States
(12) Patent Application Publication
(10) Pub. No.: US 2011/0104281 A1
(43) Pub. Date: May 5, 2011

(54) METHOD FOR TREATING PAIN USING A SUBSTITUTED 2-AMINOTETRALIN COMPOUND

(75) Inventors: Bettina Beyreuther, Dusseldorf (DE); Dieter Scheller, Neuss (DE); Joachim Freitag, Nurtingen (DE); Joseph Bianchine, Potomac, MD (US)

(73) Assignee: UCB PHARMA GMBH, Monheim (DE)

(21) Appl. No.: 12/985,058

(22) Filed: Jan. 5, 2011

Related U.S. Application Data
(62) Division of application No. 11/764,907, filed on Jun. 19, 2007, now abandoned.

(30) Foreign Application Priority Data
Jun. 22, 2006 (EP) 06 012 815.4

(51) Int. Cl. A61K 31/381; A61K 9/00; A61K 31/485; A61P 25/04; A61P 29/00

(52) U.S. Cl. 424/486; 514/438; 424/484; 514/289

(57) ABSTRACT

A method for treating pain, particularly non-inflammatory musculoskeletal pain such as myofascial pain or back pain, in a subject comprises administering to the subject a substituted 2-aminotetralin compound as defined herein, illustratively rotigotine.

### CROSS-REFERENCE TO RELATED APPLICATIONS

This application claims the benefit under 35 U.S.C. §119 of European Patent Application No. EP 06 012 815.4, filed 22 Jun. 2006, the full disclosure of which is incorporated herein by reference. This application also claims the benefit of U.S. patent application Ser. No. 11/764,907 (U.S. Publication No. 2008/0008748) filed 19 Jun. 2007.

### FIELD OF THE INVENTION

The present invention relates to methods for treatment (including prevention and/or alleviation) of various types of pain in a subject.

### BACKGROUND OF THE INVENTION

Pain is a complex physiological process that involves a number of sensory and neural mechanisms. Acute pain is typically a physiological signal indicating a potential or actual injury. Chronic pain can be somatogenic (organic) or psychogenic. Chronic pain is frequently accompanied or followed by vegetative signs, such as, for example, lassitude or sleep disturbance.

Somatogenic pain may be of nociceptive, inflammatory or neuropathic origin. Nociceptive pain is related to activation of somatic or visceral pain-sensitive nerve fibers, typically by physical or chemical injury to tissues. Inflammatory pain results from inflammation, for example an inflammatory response of living tissues to any stimulus including injury, infection or irritation. Neuropathic pain results from dysfunction in the nervous system. Neuropathic pain is believed to be sustained by aberrant somatosensory mechanisms in the peripheral nervous system, the central nervous system (CNS), or both.

Non-inflammatory musculoskeletal pain is a particular form of chronic pain that is generally not traced to a specific structural or inflammatory cause and that generally does not appear to be induced by tissue damage and macrophage infiltration (resulting in edema) as occurs in a classical immune system response.

Although non-inflammatory musculoskeletal pain is believed to result from peripheral and/or central sensitization, the cause is not presently fully understood. It is often associated with physical or mental stress, lack of adequate or restful sleep, or exposure to cold or damp. Non-inflammatory musculoskeletal pain is also believed to be associated with or precipitated by systemic disorders such as viral or other infections. Examples of non-inflammatory musculoskeletal pain include neck and shoulder pain and spasms, low back pain, and achy chest or thigh muscles. Non-inflammatory musculoskeletal pain may be generalized or localized. Understanding of the basic causes and mechanisms, animal and other models for studying non-inflammatory musculoskeletal pain, and treatment regimens are all areas where a need for improvement exists.

Fibromyalgia syndrome (FMS) and myofascial pain syndrome (MPS) are medical conditions characterized by fibromyalgia and myofascial pain respectively, which are two types of non-inflammatory musculoskeletal pain.

FMS is a complex syndrome associated with significant impairment of quality of life and can result in substantial financial costs. Fibromyalgia is a systemic process that typically causes tender points (local tender areas in normal-appearing tissues) in particular areas of the body and is frequently associated with a poor sleep pattern and/or stressful environment. Diagnosis of fibromyalgia is typically based on a history of widespread pain (e.g., bilateral, upper and lower body, and/or spinal pain), and presence of excessive tenderness on applying pressure to a number of (sometimes more precisely defined as at least 11 out of 18) specific muscle-tender sites. FMS is typically a chronic syndrome that causes pain and stiffness throughout the tissues that support and move the bones and joints.

Treatment of fibromyalgia is conventionally based on pain relievers, non-steroidal anti-inflammatory drugs (NSAIDs), muscle relaxants, tranquilizers and antidepressants, none of which are universally effective.

Fibromyalgia patients often sleep poorly and may experience some relief by taking an antidepressant such as amitriptyline at bedtime. See Goldenberg et al., J. Am. Med. Assoc. 292(19):2388-2395 (2004).

A goal in treating fibromyalgia is to decrease pain and to increase function. Fibromyalgia has been reviewed, for example by Nampiaparampil & Shmerling, Am. J. Manag. Care 10 (11 Pt 1):794-800 (2004).

Myofascial pain syndrome (MPS) is a chronic non-degenerative, non-inflammatory musculoskeletal condition often associated with spasm or pain in the masticatory muscles. Distinct areas within muscles or their delicate connective tissue coverings (fascia) become abnormally thickened or tight. When the myofascial tissues tighten and lose their elasticity, the ability of neurotransmitters to send and receive messages between the brain and body is disrupted. Specific discrete areas of muscle may be tender when firm fingertip pressure is applied; these areas are called tender or trigger points. (Both areas are tender, but trigger points additionally radiate the pain to a distant site.) Symptoms of MPS include muscle stiffness and aching and sharp shooting pains or tingling and numbness in areas distant from a trigger point. The discomfort may cause sleep disturbance, fatigue and depression. Most commonly trigger points are in the jaw (temporomandibular) region, neck, back or buttocks.

Myofascial pain differs from fibromyalgia: MPS and FMS are two separate entities, each having its own pathology, but sharing the muscle as a common pathway of pain. Myofascial pain is typically a more localized or regional (along the muscle and surrounding fascia tissues) pain process that is often associated with trigger point tenderness. Myofascial pain can be treated by a variety of methods (sometimes in combination) including stretching, ultrasound, ice sprays with stretching, exercises, and injections of anesthetic.

A further non-inflammatory musculoskeletal pain condition is back pain, notably low back pain. Back pain is a common musculoskeletal symptom that may be either acute or chronic. It may be caused by a variety of diseases and disorders that affect the lumbar spine. Low back pain is often accompanied by sciatica, which is pain that involves the sciatic nerve and is felt in the lower back, the buttocks, and the backs of the thighs.

Non-inflammatory musculoskeletal pain such as fibromyalgia, myofascial pain and back pain involves increased muscle sensitivity as an important manifestation. Increased muscle sensitivity is characterized by pain evoked by a normally non-nociceptive stimulus (allodynia) or increased pain intensity evoked by nociceptive stimuli (hyperalgesia). The term “allodynia” refers to a normally innocuous somatosensory stimulation that evokes abnormal intense pain sensation with an explosive, radiating character often outlasting stimulus or trigger duration (i.e., pain due to a stimulus that does not normally provoke pain). The term “hyperalgesia” refers to a noxious stimulation that evokes more intense and prolonged pain sensations (i.e., an increased response to a stimulus that is normally painful).

Two classes of drugs are generally employed for treatment of various types of pain: non-opioid analgesics, including acetaminophen and NSAIDs, and opioid (narcotic) analgesics. Both opioids and non-opioids have several unwanted side effects. The most serious effects of opioids are the possibility of inhibition of the respiratory system and, after long-term treatment, the possibility of addiction. NSAIDs, on the other hand, can induce a variety of gastrointestinal complications such as ulcers and bleeding, but also kidney damage.

In part because of such side effects, alternative drug therapies have been proposed for treatment of pain. Such drugs include anticonvulsants, antidepressants, serotonin modulators, norepinephrine re-uptake inhibitors, dopamine agonists and combinations thereof.

For example, U.S. Pat. No. 5,658,955 to Hitzig proposes use of a combination of a serotonin agonist and a dopamine agonist to treat fibromyalgia, among other conditions. Phentermine is described therein as a preferred dopamine agonist.

U.S. Pat. No. 5,872,127 to Cincotta & Meier proposes treatment of a variety of diseases, including fibromyalgia, through management of prolactin levels using a serotonin agonist and a dopamine agonist.

U.S. Pat. No. 6,448,258 to McCall et al. proposes treatment of fibromyalgia syndrome or chronic fatigue syndrome with compounds said to have dopamine receptor activity, including cabergoline.

The publications individually cited below each propose a method for treatment of human patients afflicted with fibromyalgia, using non-ergolinic dopamine receptor agonists which are tetrahydrobenzothiazole and 3(H)-indolone compounds, illustratively pramipexole and ropinirole respectively.

International Patent Publication No. WO 02/05797.

U.S. Pat. No. 6,277,875 to Holman.

U.S. Pat. No. 6,300,365 to Holman.

Holman, J. Musculoskeletal Pain 12(1):69-74 (2004).

Pramipexole and ropinirole are non-ergolinic agonists of the D2 subfamily of dopamine receptors (D2, D3 and D4), having strongest affinity for D3. They show only weak or no affinity for D1, for 5-hydroxytryptamine (5-HT) receptors such as 5-HT_{1A} and 5-HT₇, or for alpha-adrenergic receptors such as α2B or α2C. Pramipexole and ropinirole have been shown to reduce pain in preliminary clinical studies with fibromyalgia patients. See, for example, the publications individually cited below.

Holman, Arthritis & Rheumatism 50 (Suppl. 9):5698 (2004).

Holman et al., Arthritis & Rheumatism 52(8):2495-2505 (2005).

These dopamine agonists are known to commonly lead, usually in the beginning of therapy and as a function of the dosage administered, to various side effects including, for example, psychiatric, neurological, vascular and gastrointestinal effects. Psychiatric effects reported for pramipexole or ropinirole have included insomnia, hallucinations and confusion. Neurological effects have included syncope or fainting, somnolence, dizziness or vertigo, and dyskinesia. Gastrointestinal effects have included vomiting, nausea, abdominal pain, constipation and heartburn.

Attacks of drowsiness have been described as a serious side effect of pramipexole. For side effects of pramipexole, see, for example, a Scientific Discussion posted by the European Medicines Agency at http://www.emea.eu.int/humandocs/PDFs/EPAR/Sifrol/059197EN6.pdf.

There is a continuing need to provide alternative medicines for treatment, including systemic treatment, of chronic and/or acute pain, especially non-inflammatory musculoskeletal pain, and related conditions, in particular fibromyalgia, myofascial pain and back pain. Specifically, there is a continuing need for new treatments, including systemic treatments, for medical conditions characterized by increased pain intensity evoked by nociceptive stimuli (hyperalgesia) and/or by increased pain intensity evoked by normally non-nociceptive stimuli (allodynia) in the absence of a physiological cause such as inflammatory edema.

### SUMMARY OF THE INVENTION

It has now been found that rotigotine, representative of compounds of Formula (I) below, has analgesic properties. Such compounds can therefore be used to treat (including to prevent and/or alleviate) various types of pain. In particular, such compounds can be used to provide antinociceptive effects, more particularly to reduce muscular hyperalgesia and/or muscular allodynia, in a subject suffering from, or in anticipation of, non-inflammatory musculoskeletal pain such as back pain, fibromyalgia or myofascial pain.

Accordingly, there is now provided a method for treating pain in a subject, comprising administering to the subject a therapeutically effective amount of a compound of Formula (I)

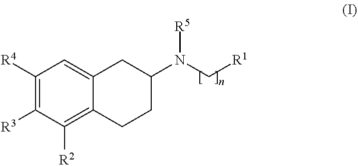

or an enantiomer, mixture of enantiomers, pharmaceutically acceptable salt, prodrug or metabolite thereof, wherein:
n is a number from 1 to 5;
R¹ is selected from the group consisting of hydrogen, 3-pyridyl, 4-pyridyl, optionally substituted phenyl,

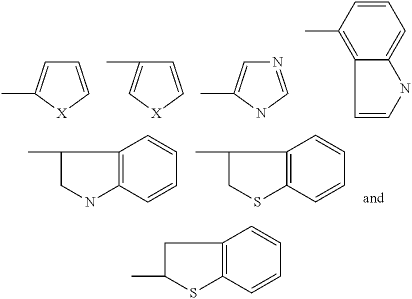

wherein X is S, O or NH;
R² is a group —OA; and
R³ and R⁴ are each independently hydrogen or a group —OA, wherein A is hydrogen;
   alkyl, in particular C₁₋₆ alkyl; cycloalkyl, in particular C₃₋₁₀ cycloalkyl; aryl, in particular optionally substituted phenyl; alkoxyalkyl, in particular alkoxy-C₁₋₆ alkyl, more particularly alkoxy-C₁₋₃ alkyl, for example alkoxymethyl; —C(═S)R⁶; —C(═S)OR⁶; —C(═S)NR⁶R⁷, for example —C(═S)NHR⁶ or —C(═S)NH₂; —S(O)₂R⁶; —S(O)₂OR⁶; —P(O₂H)R⁶; —P(O₂H)OR⁶; —CHR⁶OC(O)R⁷; —C₁₋₃ alkyl-OC(O)R⁶, in particular —CH₂—OC(O)R⁶; —C(OR⁶)R⁷R⁸, for example —CH(OR⁶)R⁷; —C(O)R⁶; —C(O)NR⁶R⁷, for example —C(O)NHR⁶ or —C(O)NH₂; or —C(O)OR⁶; wherein R⁶, R⁷ and R⁸ are each independently hydrogen; alkyl, in particular C₁₋₂₀ alkyl and more particularly C₁₋₁₂ alkyl, for example C₁₋₆ alkyl; cycloalkyl, in particular C₃₋₁₀ cycloalkyl and more particularly C₄₋₈ cycloalkyl, for example C₄₋₆ cycloalkyl; or aryl, in particular optionally substituted phenyl; and wherein alkyl substituents are optionally substituted with one or more halogen atoms; and
R⁵ is C₁₋₃ alkyl.

The compounds useful herein can be pure or substantially pure enantiomers (R or S) or any mixture thereof, including racemates, or pharmaceutically acceptable salts, prodrugs or metabolites thereof.

In an illustrative embodiment, the method comprises administering a compound of Formula (I) wherein

n is 2;

R¹ is 2-thienyl;

R² is hydroxy;

R³ and R⁴ are each hydrogen; and

R⁵ is n-propyl;

or an enantiomer, mixture of enantiomers, pharmaceutically acceptable salt, prodrug or metabolite thereof. Such a compound can be, for example, the (S)-enantiomer of (−)-5,6,7,8-tetrahydro-6-[propyl-[2-(2-thienyl)ethyl]amino]-1-naphthol, also known as rotigotine or SPM-962, or a pharmaceutically acceptable salt, prodrug or metabolite thereof.

Any of a known variety of painful medical conditions can be treated by a method of the invention. The pain to be treated can be chronic or acute and, in a particular nonlimiting example, is musculoskeletal pain, more particularly non-inflammatory musculoskeletal pain such as fibromyalgia, myofascial pain or back pain.

A related embodiment of the invention provides use of a compound of Formula (I) for the preparation of a pharmaceutical composition for treatment (including prevention and/or alleviation) of chronic and/or acute pain, for example musculoskeletal pain, more particularly non-inflammatory musculoskeletal pain such as fibromyalgia, myofascial pain or back pain.

There is further provided a method for reducing muscular hyperalgesia and/or muscular allodynia, comprising administering to the subject a therapeutically effective amount of a compound of Formula (I), for example rotigotine, or an enantiomer, mixture of enantiomers, pharmaceutically acceptable salt, prodrug or metabolite thereof.

A method of the invention can optionally further comprise administering a further active agent in combination or adjunctive therapy with a compound of Formula (I), for example rotigotine, or an enantiomer, mixture of enantiomers, pharmaceutically acceptable salt, prodrug or metabolite thereof. The further active agent can comprise one or more drugs selected from analgesics, CGRP antagonists, NMDA receptor blockers, cannabinoids, NSAIDs, COX-2 selective inhibitors, bradykinin antagonists, sedatives, antidepressants, tranquilizers and neuroprotective agents.

### BRIEF DESCRIPTION OF THE DRAWINGS

FIG. 1 presents in graphical form results of the study of Example 1, showing a dose-dependent effect of rotigotine (0.3, 1 and 3 mg/kg) by comparison with vehicle on duration of formalin-induced licking at various times after formalin injection. A star (★) indicates a significant difference from vehicle (ANOVA corrected for multiple comparisons, P<0.05).

FIG. 2 presents in graphical form results of the study of Example 1, showing a dose-dependent effect of rotigotine (0.3, 1 and 3 mg/kg) by comparison with vehicle on duration of formalin-induced licking during two phases (0-5 minutes and 10-30 minutes after formalin injection).

FIG. 3 presents in graphical form results of the study of Example 2 comparing percent maximal possible effect (% MPE) of rotigotine (0.3, 1 and 3 mg/kg) and metamizol (2 mg/kg) by comparison with vehicle (PBS) on withdrawal pressure. ★P<0.05 (ANOVA+Bonferroni post hoc) versus PBS.+P<0.05 (Mann-Whitney-U test) versus PBS.

FIG. 4 presents in graphical form results of the study of Example 2 comparing percent maximal possible effect (% MPE) of rotigotine (0.3, 1 and 3 mg/kg) and metamizol (2 mg/kg) by comparison with vehicle (PBS) on grip force. ++P<0.01 (Mann-Whitney-U test) versus PBS.

### DETAILED DESCRIPTION

As indicated above, the present invention involves administration of a substituted 2-aminotetralin compound of Formula (I)

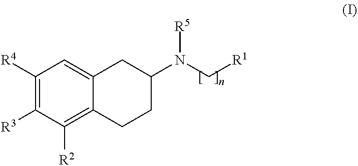

or an enantiomer, mixture of enantiomers, pharmaceutically acceptable salt, prodrug or metabolite thereof.

In Formula (I), n is a number from 1 to 5, illustratively 1 to 3, more particularly 2 or 3, for example 2.

R¹ in Formula (I) is selected from the group consisting of hydrogen, 3-pyridyl, 4-pyridyl, optionally substituted phenyl,

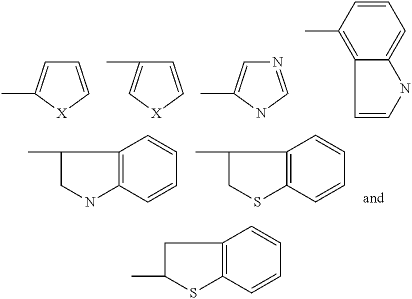

wherein X is S, O or NH.

Illustratively, R¹ is

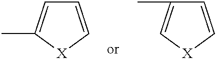

wherein X is as defined above, more particularly a sulfur atom. For example, R¹ can be 2-thienyl.

R² in Formula (I) is a group —OA, wherein A is hydrogen; alkyl, in particular C₁₋₆ alkyl; cycloalkyl, in particular C₃₋₁₀ cycloalkyl; aryl, in particular optionally substituted phenyl; alkoxyalkyl, in particular alkoxy-(C₁₋₆ alkyl), more particularly alkoxy-(C₁₋₃ alkyl), for example alkoxymethyl; —C(═S)R⁶; —C(═S)OR⁶; —C(═S)NR⁶R⁷, for example —C(═S)NHR⁶ or —C(═S)NH₂; —S(O)₂R⁶; —S(O)₂OR⁶; —P(O₂H)R⁶; —P(O₂H)OR⁶; —CHR⁶—O—C(O)R⁷; —C₁₋₃ alkyl-O—C(O)R⁶; —C(OR⁶)R⁷R⁸, for example —CH(OR⁶)R⁷; —C(O)R⁶; —C(O)NR⁶R⁷, for example —C(O)NHR⁶ or —C(O)NH₂; or —C(O)OR⁶; wherein R⁶, R⁷ and R⁸ are each independently hydrogen; alkyl, in particular C₁₋₂₀ alkyl, more particularly C₁₋₆ alkyl; cycloalkyl, in particular C₃₋₁₀ cycloalkyl; or aryl, in particular optionally substituted phenyl. Alkyl groups are optionally substituted with one or more halogen atoms, but are illustratively unsubstituted.

Illustratively, R² is a group —OA, wherein A is hydrogen or a group

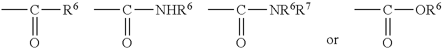

wherein R⁶ and R⁷ are each independently a C₁₋₂₀ alkyl (in particular C₁₋₁₂ alkyl, more particularly C₁₋₆ alkyl), phenyl or methoxyphenyl group. For example, R² can be —OH or —OC(O)CH₃.

R³ and R⁴ in Formula (I) are each independently hydrogen or —OA, wherein A is as defined above, illustratively hydrogen or a group

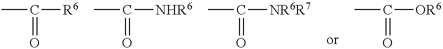

wherein R⁶ and R⁷ are each independently a C₁₋₂₀ alkyl (in particular C₁₋₁₂ alkyl, more particularly C₁₋₆ alkyl), phenyl or methoxyphenyl group.

In one embodiment, R³ is hydrogen.

In one embodiment, R⁴ is hydrogen.

In one embodiment, R³ and R⁴ are each hydrogen.

In one embodiment, R³ and R⁴ are each hydrogen, R² is —OH or —OC(O)CH₃, and n is 2.

R⁵ in Formula (I) is C₁₋₃ alkyl, for example C₃ alkyl, in particular n-propyl.

In one embodiment, R¹ is 2-thienyl, R³ and R⁴ are each hydrogen, R⁵ is C₃ alkyl and n is 2.

In one embodiment, a method of the invention comprises administering a compound of Formula (I)

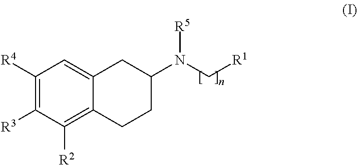

or an enantiomer, mixture of enantiomers, pharmaceutically acceptable salt, prodrug or metabolite thereof, wherein:
n is a number from 1 to 3;
R¹ is

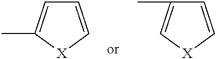

wherein X is O, S or NH;
R² is a group —OA, and R³ and R⁴ are each independently hydrogen or a group —OA,
   wherein A is hydrogen or a group

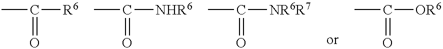

wherein R⁶ and R⁷ are each independently a C₁₋₂₀ alkyl (in particular C₁₋₁₂ alkyl, more particularly C₁₋₆ alkyl), phenyl or methoxyphenyl group; and
R⁵ is C₁₋₃ alkyl.

In a particular embodiment, n is 2; R¹ is 2-thienyl; R² is hydroxy; R³ and R⁴ are each hydrogen; and R⁵ is n-propyl. The compound of Formula (I) in this case is 5,6,7,8-tetrahydro-6-[propyl-[2-(2-thienyl)ethyl]amino]-1-naphthol or an enantiomer, mixture of enantiomers, pharmaceutically acceptable salt, prodrug or metabolite thereof.

Compounds of Formula (I), where optically active as in the case of 5,6,7,8-tetrahydro-6-[propyl-[2-(2-thienyl)ethyl]amino]-1-naphthol, can be present as mixtures of enantiomers, for example racemates, or as pure (R)- or (S)-enantiomers. The term “pure enantiomer” herein means that at least about 90 mol % of the compound in question is present in the form of one enantiomer, e.g., in the (S) form, while the proportion of the respective other enantiomer, e.g., the (R) form, is correspondingly low.

Rotigotine (SPM-962) is the (S)-(−)-enantiomer of 5,6,7,8-tetrahydro-6-[propyl-[2-(2-thienyl)ethyl]amino]-1-naphthol. Rotigotine used according to the present method typically is the pure (S)-(−)-enantiomer; the corresponding (R)-(+)-enantiomer typically represents less than about 10 mol %, more particularly less than about 2 mol %, for example less than about 1 mol %, of the total amount of 5,6,7,8-tetrahydro-6-[propyl-[2-(2-thienyl)ethyl]amino]-1-naphthol in the pharmaceutical composition.

Compounds of Formula (I) can be present as free bases and/or in the form of pharmaceutically acceptable salts, e.g., rotigotine in the form of rotigotine hydrochloride. Pharmaceutically acceptable salts include non-toxic addition salts of a compound of Formula (I) with organic or inorganic acids. Examples of inorganic acids include HCl.

The terms “C₁₋₂₀ alkyl”, “C₁₋₁₂ alkyl”, “C₁₋₆ alkyl” and “C₁₋₃ alkyl” as used herein mean, independently of each other, branched or unbranched alkyl groups with a total number of carbon atoms in the corresponding range. A “C₁₋₂₀ alkyl” group has, for example, 1 to 20 carbon atoms (a numerical range herein is always inclusive of the lowest and highest values stated). Alkyl groups can optionally be substituted, e.g., with halogen. In a particular embodiment the alkyl groups are unsubstituted.

The term “cycloalkyl” when used alone or in combination means a cycloalkyl group containing from 3 to 18 ring carbon atoms and up to a total of 25 carbon atoms. Cycloalkyl groups may be monocyclic, bicyclic, tricyclic or polycyclic and the rings can be fused. Cycloalkyl groups may be completely or partially saturated. Examples include cyclopropyl, cyclobutyl, cyclopentyl, cyclohexyl, cycloheptyl, cyclooctyl, cyclodecyl, cyclohexenyl, cyclopentenyl, cyclooctenyl, cycloheptenyl, decalinyl, hydroindanyl, indanyl, fenchyl, pinenyl, adamantyl and the like. Cycloalkyl includes the cis- and trans- forms. Cycloalkyl groups may be unsubstituted or mono- or polysubstituted with electron-withdrawing and/or electron-donating groups as described below. Furthermore, such substituents if present may be in endo- or exo-positions in bridged bicyclic systems. Illustrative cycloalkyl groups include those with 3 to 10, in particular 4 to 8, more particularly 4 to 6 ring carbon atoms.

The term “alkoxy” herein means lower alkoxy containing from 1 to 6, especially 1 to 3 carbon atoms, that may be straight chain or branched. Alkoxy groups include methoxy, ethoxy, propoxy, butoxy, isobutoxy, tert-butoxy, pentoxy, hexoxy and the like.

The term “aryl”, when used alone or in combination, refers to an aromatic group which contains from 6 to 18 ring carbon atoms and up to a total of 25 carbon atoms, and includes polynuclear aromatics. Aryl groups may be monocyclic, bicyclic, tricyclic or polycyclic and can comprise fused rings. Polynuclear aromatic groups herein encompass bicyclic and tricyclic fused aromatic ring systems containing from 10 to 18 ring carbon atoms and up to a total of 25 carbon atoms. Aryl groups include phenyl and polynuclear aromatic groups such as naphthyl, anthracenyl, phenanthrenyl, azulenyl and the like, and also include groups such as ferrocenyl. Aryl groups may be unsubstituted or mono- or polysubstituted with electron-withdrawing and/or electron-donating groups as described below. In one embodiment an aryl group is a phenyl group.

The terms “electron-withdrawing” and “electron-donating” refer to the ability of a substituent to withdraw or donate electrons, respectively, relative to that of hydrogen if a hydrogen atom occupied the same position in the molecule. These terms are well understood by one skilled in the art and are discussed, for example, in March, Advanced Organic Chemistry, New York, John Wiley and Sons (1985), at pp. 16-18, the disclosure of which is incorporated herein by reference. Electron-withdrawing groups include halo (including fluoro, chloro, bromo and iodo), nitro, carboxy, lower alkenyl, lower alkynyl, formyl, carboxyamido, aryl, quaternary ammonium, haloalkyl (such as trifluoromethyl), aryl lower alkanoyl, carbalkoxy, and the like. Electron-donating groups include hydroxy, lower alkoxy (including methoxy, ethoxy, and the like), lower alkyl (including methyl, ethyl, and the like), amino, lower alkylamino, di(lower alkyl)amino, aryloxy (such as phenoxy), mercapto, lower alkylthio, lower alkylmercapto, disulfide (lower alkyldithio), and the like. One of ordinary skill in the art will appreciate that some of the aforesaid substituents may be considered to be electron-donating or electron-withdrawing under different chemical conditions. Moreover, the present invention contemplates any combination of substituents selected from the above-identified groups.

Illustrative electron-donating and/or electron-withdrawing substituents are halo, nitro, alkanoyl, formyl, arylalkanoyl, aryloyl, carboxyl, carbalkoxy, carboxamido, cyano, sulfonyl, sulfoxide, heterocyclic, guanidine, quaternary ammonium, lower alkenyl, lower alkynyl, sulfonium salts, hydroxy, lower alkoxy, lower alkyl, amino, lower alkylamino, di(lower alkyl)amino, amino lower alkyl, mercapto, mercaptoalkyl, alkylthio, and alkyldithio. The term “sulfide” encompasses mercapto, mercapto alkyl and alkylthio, while the term “disulfide” encompasses alkyldithio. Particular examples of electron-donating and/or electron-withdrawing groups are halo and lower alkoxy, such as fluoro or methoxy.

In a further embodiment, the compound administered is a prodrug of an active compound of Formula (I), for example such a compound wherein R¹ is 2-thienyl, R³ and R⁴ are each hydrogen, R⁵ is C₃ alkyl, n is 2, and R² is —OA, wherein A is a chemical moiety as defined above, more particularly wherein the active compound is rotigotine.

A prodrug is an agent that generally has weak or no pharmaceutical activity itself but is converted into a pharmaceutically active compound in vivo. Prodrugs are often useful because, in some situations, they may be easier to administer than the corresponding active compound. A prodrug may, for instance, be bioavailable by oral administration where the active compound is not. A prodrug may be simpler to formulate, for example through improved solubility in a pharmaceutical composition, than the active compound. Conventional procedures for selection and preparation of suitable prodrug derivatives are described, for example, in the publications individually cited below.

Bundgaard, ed., Design of prodrugs. New York, N.Y.: Elsevier (1985).

Higuchi & Stella, eds., Prodrugs as novel drug delivery systems. Washington, D.C.: American Chemical Society (1975).

Sloan, ed., Prodrugs, topical and ocular drug delivery. New York, N.Y.: Marcel Dekker (1992).

Roche, ed. Design of biopharmaceutical properties through prodrugs and analogs, Washington, D.C.: American Pharmaceutical Society (1977).

As a nonlimiting example, prodrugs useful herein can be derivatives of a compound of Formula (I) such as rotigotine at the phenolic hydroxy group thereof.

Illustrative prodrugs of rotigotine are described, for example, in the publications individually cited below and incorporated herein by reference.

Den Daas et al., Naunyn Schiedebergs Arch. Pharmacol. 341:186-191 (1990).

Den Daas et al., J. Pharm. Pharmacol. 43:11-16 (1991).

The suitability of a prodrug of a compound of Formula (I), for example rotigotine, can for example be determined by incubating a particular prodrug candidate under defined conditions with an enzyme cocktail and a cell homogenizate or an enzyme-containing cell fraction, and measuring the active compound such as rotigotine. A suitable enzyme mixture is for example the S9 liver preparation distributed by Gentext of Woburn, Mass. Other methods to test the suitability of a prodrug of a compound of Formula (I), for example rotigotine, are known to one skilled in the art.

For example, in vitro conversion of a prodrug into the active substance can be assayed in the following way. The microsome fraction containing essential metabolic enzymes is obtained from liver cell homogenizates from humans, monkeys, dogs, rats and/or mice by differential centrifugation; alternatively, it is also possible to obtain the cytoplasmic fraction. The subcellular fraction is suspended with a buffer in such a way that a solution with a defined protein content is obtained. After the addition of 1 μM of the prodrug to be tested, it is incubated at 37° C. for 60 minutes. Then rotigotine is quantified by means of HPLC/UV or HPLC/MS and related to the quantity used. For more detailed analyses, concentration or time series are investigated.

In a further embodiment, the compound administered is a metabolite of a compound of Formula (I), for example such a compound wherein R¹ is 2-thienyl, R³ and R⁴ are each hydrogen, R⁵ is C₃ alkyl, n is 2, and R² is —OA, wherein A is a chemical moiety as defined above, more particularly wherein the active compound is rotigotine. An example of such a metabolite of rotigotine is (S)-2-N-propylamino-5-hydroxytetralin, as disclosed for example in International Patent Publication No. WO 2005/058296, incorporated herein by reference.

Substituted 2-aminotetralin compounds useful herein, such as rotigotine, can be prepared in a conventional fashion, for example as described in European Patent No. EP 0 168 505, incorporated herein by reference.

Analgesic potency of compounds of Formula (I) can be demonstrated, for example, using the validated animal models described in Examples 1 and 2 herein.

Compounds to be used according to the present disclosure, such as rotigotine and the like, have analgesic properties, making them suitable for administration to a subject for treatment (including prevention and/or alleviation) of chronic and/or acute pain, in particular non-inflammatory musculoskeletal pain such as back pain, fibromyalgia and myofascial pain, more particularly for reduction of the associated muscular hyperalgesia or muscular allodynia. In particular, the compounds of Formula (I), more particularly rotigotine, are used for the preparation of a pharmaceutical composition for the prevention, alleviation and/or treatment of fibromyalgia.

Nonlimiting examples of types of pain that can be treated by the method of the present disclosure are chronic conditions such as musculoskeletal pain, including fibromyalgia, myofascial pain, back pain, pain during menstruation, pain during osteoarthritis, pain during rheumatoid arthritis, pain during gastrointestinal inflammation, pain during inflammation of the heart muscle, pain during multiple sclerosis, pain during neuritis, pain during AIDS, pain during chemotherapy, tumor pain, headache, CPS (chronic pain syndrome), central pain, neuropathic pain such as trigeminal neuralgia, shingles, stamp pain, phantom limb pain, temporomandibular joint disorder, nerve injury, migraine, post-herpetic neuralgia, neuropathic pain encountered as a consequence of injuries, amputation infections, metabolic disorders or degenerative diseases of the nervous system, neuropathic pain associated with diabetes, pseudesthesia, hypothyroidism, uremia, vitamin deficiency or alcoholism; and acute pain such as pain after injuries, postoperative pain, pain during acute gout or pain during operations, such as jaw surgery.

In a particular embodiment, a compound of Formula (I), for example rotigotine, is administered for treatment of non-inflammatory musculoskeletal pain such as fibromyalgia (e.g., in FMS), myofascial pain (e.g., in MPS) or back pain, and in particular for reducing muscular hyperalgesia or muscular allodynia associated with such conditions. In a more particular embodiment, the condition to be treated is fibromyalgia.

In another embodiment a compound of Formula (I), for example rotigotine, is administered for treatment of neuropathic pain.

Unless the context demands otherwise, the term “treat,” “treating” or “treatment” herein includes preventive or prophylactic use of an agent, for example a compound of Formula (I), in a subject at risk of pain, having a prognosis including pain, or having a condition or syndrome such as FMS or MPS characterized by recurrent pain, as well as use of such an agent in a subject already experiencing pain, as a therapy to alleviate, relieve, reduce intensity of or eliminate such pain or an underlying cause thereof. The standard of care in treating chronic pain is to administer an analgesic agent in anticipation of recurrence of pain, as opposed to allowing the pain to recur before giving further treatment. See for example Grahame-Smith & Aronson, eds., Oxford Textbook of Clinical Pharmacology and Drug Therapy, 2nd ed. Oxford University Press (1992), p. 460.

The term “subject” refers to a warm-blooded animal, generally a mammal such as, for example, a cat, dog, horse, cow, pig, mouse, rat or primate, including a human. In one embodiment the subject is a human, for example a patient having or at risk of a pain condition such as fibromyalgia, myofascial pain or back pain.

The term “central pain” refers to pain associated with a lesion of the central nervous system.

In one embodiment, the compound, for example rotigotine, is administered to a subject suffering from pain, for example one of the types of pain mentioned above, such as fibromyalgia, myofascial pain or back pain, in an analgesic effective amount. The term “effective amount” as used herein means an amount of a compound effective to result in a clinically determinable improvement in, or suppression of, symptoms associated with a medical condition. An improvement in such symptoms can include, in the case of pain symptoms, reduction in intensity, reduction in frequency, or complete cessation of pain for a sustained period of time. An analgesic effective amount for such a subject is equivalent to a therapeutically effective amount as described herein.

A substituted 2-aminotetralin compound of Formula (I), for example rotigotine, can be used alone or in a pharmaceutical composition together with a pharmaceutically acceptable carrier.

There are many methods of application available for administering substituted 2-aminotetralins of Formula (I), in particular rotigotine, which the person skilled in the art can select and adapt depending on the need, condition and age of the subject, the required dosage and the desired application interval. As nonlimiting examples, the route of administration can be parenteral, transdermal or transmucosal.

In one particular embodiment, the route of administering a substituted 2-aminotetralin compound of Formula (I), for example rotigotine, is transdermal administration. The form and pharmaceutical composition in which the compound is administered is adapted for the route of administration and, in the case of transdermal administration, a suitable composition can be, for example, an ointment, a gel, a cream, a paste, a spray, a film, a plaster, a patch, a poultice, a cataplasm or an iontophoretic device.

Illustratively according to this embodiment, a substituted 2-aminotetralin of Formula (I), for example rotigotine, may be administered by application to a patient's skin of a patch or plaster having the active substance present in an adhesive polymer matrix, for instance a self-adhesive polysiloxane matrix. Examples of suitable transdermal formulations can be found in the publications individually cited below and incorporated herein by reference.

International Patent Publication No. WO 99/49852.

International Patent Publication No. WO 02/89777.

International Patent Publication No. WO 02/89778.

Such a method of administration can enable a substantially constant plasma level to be established and therefore a substantially constant dopaminergic stimulation over an entire application interval. See Metman, Clin. Neuropharmacol. 24:163 (2001). Further, constant delivery by transdermal administration can result in a rapid achievement of a desired dose, particularly by comparison with pulsatile administration of a compound.

If, on the other hand, administration in the form of a subcutaneous or intramuscular depot is desired, a substituted 2-aminotetralin compound of Formula (I), for example rotigotine, may be suspended, for example as salt crystals such as crystalline rotigotine hydrochloride, in a hydrophobic anhydrous medium, for administration by injection, as described for example in International Patent Publication No. WO 02/15903, incorporated herein by reference.

Otherwise, the compound may be administered in the form of microcapsules, microparticles or implants based on biodegradable polymers, as described, for example, in International Patent Publication No. WO 02/38646, incorporated herein by reference.

Other dosage forms suitable for administering a substituted 2-aminotetralin of Formula (I), for example rotigotine, are transmucosal formulations, for example sublingual sprays, rectal formulations or aerosols for pulmonary administration.

Suitable dosages of substituted 2-aminotetralins of Formula (I), in particular rotigotine, are typically about 0.05 to about 50 mg/day, for example about 0.1 to about 40 mg/day, about 0.2 to about 20 mg/day or about 4 to about 20 mg/day. Optionally, gradually increasing dosages can be administered, i.e., treatment can optionally start with low doses which are incrementally increased until a maintenance dose is reached.

It is clear to the person skilled in the art that the dosage interval may vary depending on the applied quantity, the mode of administration and the daily requirement of the patient or subject. Thus, a transdermal form of application may be designed, e.g., for administration once a day, once every three days or once every seven days, while a subcutaneous or intramuscular depot can make it possible to administer injections, e.g., in once weekly, bi-weekly, or monthly cycles.

The term “transdermal therapeutic system”, or its abbreviation “TTS”, as used herein refers to a pharmaceutical composition, in a form of one to a plurality of patch or plaster formulations, that contains an active agent, for example a compound of Formula (I) such as rotigotine, and that when applied to skin of a subject delivers at least a portion of the active agent into and across the skin, where the active agent accesses the circulatory system of the subject. A TTS useful herein can be prepared by processes known in the art, for example as described in the publications individually listed below and incorporated herein by reference.

U.S. Pat. No. 6,562,363 to Mantelle et al.

U.S. Pat. No. 6,884,434 to Muller & Peck.

U.S. Patent Application Publication No. 2003/0026830 of Lauterback et al.

U.S. Patent Application Publication No. 2003/0027793 of Lauterbach et al.

U.S. Patent Application Publication No. 2004/0081683 of Schacht et al.

U.S. Patent Application Publication No. 2005/0019385 of Houze.

U.S. Patent Application Publication No. 2005/0079206 of Schacht et al.

U.S. Patent Application Publication No. 2006/0216336 of Wolff, not admitted to be prior art to the present invention.

A TTS useful herein is illustratively of a reservoir or matrix type comprising one or more layers. Typically the TTS has on one side a backing layer and on the opposing side a liner layer that can be removed to expose an adhesive surface or layer that in use contacts the skin surface. The active agent can be distributed, for example as a solution or dispersion, in a matrix formed by the adhesive layer, or it can be present in a separate reservoir layer. The following description of an illustrative matrix-type TTS refers specifically to rotigotine as the active agent but it will be understood that a different compound of Formula (I) or an enantiomer, mixture of enantiomers, pharmaceutically acceptable salt, prodrug or metabolite thereof can be substituted if desired. Such a TTS can consist of one to a plurality of patches of similar composition.

Illustratively, a matrix-type TTS for administering rotigotine comprises three layers:
(1) a flexible backing sheet or layer, for example comprising an aluminized polyester foil siliconized on its inner side and coated with a pigment layer or transparent polyester film on its outer side;
(2) a matrix layer that is typically self-adhesive and contains rotigotine distributed therein; a suitable matrix layer comprises an adhesive component, e.g., comprising one or more silicone adhesives, and optionally a compatibilizing component, e.g., comprising a polymer such as povidone, a vinylpyrrolidone/vinyl acetate copolymer or an ethylene/vinyl acetate copolymer, that provides for increased concentration, homogeneity and/or stability of dispersion of the active agent in the matrix layer and/or for enhanced cohesion of the matrix layer; and
(3) a removable liner layer, for example comprising a fluoropolymer-coated polyester film.

The backing and liner layers should be inert to the components of the matrix layer.

The rotigotine can be present in free base or salt (e.g., hydrochloride salt) form or both, but where, as in the present illustrative example, the adhesive matrix is silicone-based it will be found preferable to use rotigotine that is substantially all, for example at least about 95 mol %, at least about 98 mol % or at least about 99 mol %, in free base form.

The matrix layer can be of any suitable thickness but typically is relatively thin, having a total weight of about 10 to about 100 g/m², for example about 20 to about 80 g/m² or about 40 to about 60 g/m². Rotigotine is present in the matrix layer at a concentration illustratively of about 5% to about 25%, for example about 6% to about 20%, about 7% to about 15% or about 8% to about 10%, by weight. In one embodiment, a matrix layer having a total weight of about 50 g/m² (i.e., about 5 mg/cm²) contains rotigotine free base at a concentration of about 9% by weight.

Illustratively rotigotine is present in the TTS in an amount of about 0.05 to about 2.5 mg/cm², for example about 0.1 to about 2 mg/cm², about 0.2 to about 1.5 mg/cm², about 0.3 to about 1 mg/cm² or about 0.4 to about 0.5 mg/cm². In one embodiment, rotigotine free base is present in an amount of about 0.45 mg/cm².

It will be evident that the rotigotine dose present in a TTS can be adjusted by modifying any one or more of matrix weight, rotigotine concentration in the matrix and/or surface area of the TTS. “Surface area” herein refers to the total area of one to a plurality of patches applied at one time to skin of a subject, more specifically to the area of the adhesive matrix in contact with the skin. In one embodiment a series of patches are provided having substantially similar matrix composition, weight and rotigotine concentration, but differing in surface area so as to provide a range of rotigotine dosages.

Typically, a TTS useful herein contains in total about 4 to about 20 mg rotigotine free base. Illustratively, a TTS having a surface area of about 10 cm² contains about 4.5 mg rotigotine free base; a TTS having a surface area of about 20 cm² contains about 9 mg rotigotine free base; a TTS having a surface area of about 30 cm² contains about 13.5 mg rotigotine free base; and a TTS having a surface area of about 40 cm² contains about 18 mg rotigotine free base.

In a silicone-based adhesive matrix, rotigotine free base can be present in solution up to the limit of its solubility in the matrix, but is typically also present in discrete microparticles distributed throughout the matrix. These microparticles can be of any suitable size but it is generally desirable that they be small enough to provide a substantially clear, rather than cloudy or milky, matrix layer. It is also generally desirable that the microparticles comprise rotigotine free base in an amorphous form, to avoid problems that can arise through crystal growth. Use of a compatibilizing agent such as povidone can provide improved physical stability of the matrix layer, for example by inhibiting crystallization of rotigotine. It is believed, without being bound by theory, that in a TTS having povidone in the matrix layer the microparticles comprise a stable amorphous povidone/rotigotine free base complex and act as microreservoirs of rotigotine within the matrix. Povidone is illustratively present in the matrix layer in a concentration of about 1.5% to about 5% by weight.

One or more silicone adhesives can be used in the matrix layer. Amine-resistant silicone adhesives are preferred. Suitable silicone adhesives include without limitation high-tack silicone adhesives such as BIO-PSA® Q7-4301 of Dow Corning and medium-tack silicone adhesives such as BIO-PSA® Q7-4201 of Dow Corning. In one embodiment both a high-tack and a medium-tack silicone adhesive are present, for example in a weight ratio of about 40:60 to about 60:40, illustratively about 50:50.

Other ingredients are optionally present in the matrix layer, including for example one or more antioxidants and/or antimicrobial preservatives.

An illustrative 10 cm² rotigotine patch comprises a matrix layer having the following composition:

| | |
|---|---|
| rotigotine free base | 4.50 mg |
| povidone | 1.00 mg |
| BIO-PSA ® Q7-4301 | 22.24 mg |
| BIO-PSA ® Q7-4201 | 22.23 mg |
| ascorbyl palmitate | 0.01 mg |
| DL-α-tocopherol | 0.025 mg |
| sodium metabisulfite | 0.00045 mg |

Application of one such patch provides an applied dose of 4.5 mg. Application of two, three or four such patches provides an applied dose of 9, 13.5 or 18 mg respectively.

An illustrative 20 cm² rotigotine patch comprises a matrix layer having the following composition:

| | |
|---|---|
| rotigotine free base | 9.00 mg |
| povidone | 2.00 mg |
| BIO-PSA ® Q7-4301 | 44.47 mg |
| BIO-PSA ® Q7-4201 | 44.46 mg |
| ascorbyl palmitate | 0.02 mg |
| DL-α-tocopherol | 0.05 mg |
| sodium metabisulfite | 0.0009 mg |

Application of one such patch provides an applied dose of 9 mg. Application of two such patches provides an applied dose of 18 mg.

An illustrative 30 cm² rotigotine patch comprises a matrix layer having the following composition:

| | |
|---|---|
| rotigotine free base | 13.50 mg |
| povidone | 3.00 mg |
| BIO-PSA ® Q7-4301 | 66.71 mg |
| BIO-PSA ® Q7-4201 | 66.70 mg |
| ascorbyl palmitate | 0.03 mg |
| DL-α-tocopherol | 0.075 mg |
| sodium metabisulfite | 0.00135 mg |

Application of one such patch provides an applied dose of 13.5 mg.

In each case a suitable film for the backing layer is Scotchpak® 1109.

A TTS as described above is suitable for release of rotigotine over a period of about 24 hours, but TTSs with longer or shorter release periods can be used. A TTS having a 24-hour release period as described above is suitable for administration of a daily applied dose of rotigotine of about 0.9 to about 27 mg, more typically about 4 to about 20 mg. An “applied dose” herein is the amount of rotigotine present in a TTS (whether consisting of one or a plurality of patches) administered to a subject in a day. As is generally the case with transdermal systems, not all of the active agent is released from the TTS and delivered to, i.e., received by, the subject. Illustratively, if the dose actually received by the subject is about 44% of the applied dose, a 4.5 mg, 9 mg, 13.5 mg or 18 mg applied dose is equivalent respectively to a 2 mg, 4 mg, 6 mg or 8 mg received dose.

In various embodiments, a TTS applied to skin of the subject can be removed after the release period and a further TTS applied at a suitable administration interval, for example about twice daily to about once monthly, or about once daily to about once weekly. Most typically, the TTS is replaced at an interval of about 24 to about 48 hours.

It is not necessary for the TTS to be applied to an area of the subject's body where the sensation of pain occurs. Any skin surface generally suitable for transdermal drug administration can be used as a locus for application of the TTS, including without limitation the front of the abdomen, thigh, hip, flank, shoulder or upper arm. Successive applications of a TTS can be to the same area of skin or to different areas of skin. It can be advantageous to select a different locus on successive days, for example the right side one day and the left side the next day, the upper body one day and the lower body the next day, etc. By varying or rotating the locus of application of the TTS, it will generally be possible to minimize skin irritation or other local reactions to the TTS.

In one embodiment, rotigotine is administered according to a method of the present invention by applying to skin of the subject (a) a reference TTS having a matrix layer that consists essentially of 4.5 mg rotigotine free base, 1.0 mg povidone, 22.24 mg BIO-PSA® Q7-4301 or a silicone adhesive substantially identical thereto, 22.23 mg BIO-PSA® Q7-4201 or a silicone adhesive substantially identical thereto, 0.01 mg ascorbyl palmitate, 0.025 mg DL-α-tocopherol and 0.00045 mg sodium metabisulfite per 10 cm², and having a total surface area for release of rotigotine of about 10 to about 40 cm², or (b) a rotigotine-containing TTS that is substantially bioequivalent to the reference TTS. A “substantially bioequivalent” TTS in the present context is one that exhibits, upon administration to human subjects in accordance with standard pharmacokinetic (PK) principles, a bioavailability (as measured, for example, by PK parameters including Cₘₐₓ and AUC₀₋₂₄) that is about 80% to about 125% of that exhibited by the reference TTS. PK data for a reference TTS as defined above may be determined by comparative testing in a PK study, or may be found in the literature, for example, in above-cited U.S. Patent Application Publication No. 2006/0216336, incorporated by reference herein without admission that it constitutes prior art to the present invention.

In one embodiment, a treatment method of the present invention comprises administering to the subject, for example a human subject in need of such treatment, a compound of Formula (I), for example rotigotine, in combination with administering a further active agent. The further active agent can be one effective for treatment (including prevention and/or alleviation) of chronic and/or acute pain, in particular for systemic treatment of non-inflammatory musculoskeletal pain, including specific manifestations thereof such as muscular hyperalgesia and/or allodynia, occurring in fibromyalgia, myofascial pain or back pain. The compound of Formula (I), for example rotigotine, and the further active agent may be administered together, i.e., in a single dosage form, or separately, i.e., in separate dosage forms. If administered separately, the compound of Formula (I), for example rotigotine, and the further active agent can be administered at the same or different times.

A therapeutic combination comprising a compound of Formula (I), for example rotigotine, and a further active agent as defined herein is a further embodiment of the present invention.

In a particular embodiment, a pharmaceutical composition is provided comprising a compound of Formula (I), for example rotigotine, and a further active agent effective for treatment (including prevention and/or alleviation) of chronic and/or acute pain, in particular for systemic treatment of non-inflammatory musculoskeletal pain, including specific manifestations thereof such as muscular hyperalgesia and/or allodynia, occurring in fibromyalgia, myofascial pain or back pain.

The “further active agent” mentioned above can for example be another analgesic compound such as an opioid, for example fentanyl; a calcitonin gene-related peptide (CGRP) antagonist, for example olcegepant; an N-methyl-D-aspartate (NMDA) receptor blocker, for example dextromethorphan; a cannabinoid; a bradykinin antagonist; acetaminophen; an NSAID; or a COX-2 selective inhibitor. In other embodiments the “further active agent” is for example a sedative, antidepressant, tranquilizer, neuroprotective agent, etc.

Nonlimiting examples of opioid and non-opioid analgesics that can be useful in the further active agent include acetaminophen, alfentanil, allylprodine, alphaprodine, anileridine, benzylmorphine, bezitramide, buprenorphine, butorphanol, clonitazene, codeine, cyclazocine, desomorphine, dextromoramide, dextropropoxyphene, dezocine, diampromide, diamorphone, dihydrocodeine, dihydromorphine, dimenoxadol, dimepheptanol, dimethylthiambutene, dioxaphetyl butyrate, dipipanone, dipyrone (metamizol), eptazocine, ethoheptazine, ethylmethylthiambutene, ethylmorphine, etonitazene, fentanyl, heroin, hydrocodone, hydromorphone, hydroxypethidine, isomethadone, ketobemidone, levallorphan, levorphanol, levophenacyl-morphan, lofentanil, meperidine, meptazinol, metazocine, methadone, metopon, morphine, myrophine, nalbuphine, nalorphine, narceine, nicomorphine, norlevorphanol, normethadone, normorphine, norpipanone, opium, oxycodone, oxymorphone, papavereturn, pentazocine, phenadoxone, phenazocine, phenomorphan, phenoperidine, piminodine, piritramide, proheptazine, promedol, properidine, propiram, propoxyphene, sufentanil, tilidine, tramadol, NO-naproxen, NCX-701, ALGRX-4975, pharmaceutically acceptable salts thereof, and combinations thereof.

Nonlimiting examples of NSAIDs that can be useful in the further active agent include salicylic acid derivatives (such as salicylic acid, acetylsalicylic acid, methyl salicylate, diflunisal, olsalazine, salsalate and sulfasalazine), indole and indene acetic acids (such as indomethacin, etodolac and sulindac), fenamates (such as etofenamic, meclofenamic, mefenamic, flufenamic, niflumic and tolfenamic acids), heteroaryl acetic acids (such as acemetacin, alclofenac, clidanac, diclofenac, fenchlofenac, fentiazac, furofenac, ibufenac, isoxepac, ketorolac, oxipinac, tiopinac, tolmetin, zidometacin and zomepirac), aryl acetic acid and propionic acid derivatives (such as alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid and tioxaprofen), enolic acids (such as the oxicam derivatives ampiroxicam, cinnoxicam, droxicam, lornoxicam, meloxicam, piroxicam, sudoxicam and tenoxicam, and the pyrazolone derivatives aminopyrine, antipyrine, apazone, dipyrone, oxyphenbutazone and phenylbutazone), alkanones (such as nabumetone), nimesulide, proquazone, MX-1094, licofelone, pharmaceutically acceptable salts thereof, and combinations thereof.

Nonlimiting examples of COX-2 selective inhibitors that can be useful in the further active agent include celecoxib, deracoxib, valdecoxib, parecoxib, rofecoxib, etoricoxib, lumiracoxib, PAC-10549, cimicoxib, GW-406381, LAS-34475, CS-502, pharmaceutically acceptable salts thereof, and combinations thereof.

Nonlimiting examples of NMDA receptor blockers that can be useful in the further active agent include amantadine, D-AP5, aptiganel, CPP, dexanabinol, dextromethorphan, dextropropoxyphene, 5,7-dichlorokynurenic acid, gavestinel, ifendopril, ketamine, ketobemidone, licostinel, LY-235959, memantine, methadone, MK-801, phencyclidine, remacemide, selfotel, tiletamine, pharmaceutically acceptable salts thereof, and combinations thereof.

Nonlimiting examples of sedatives that can be useful in the further active agent include without limitation acylic ureides, alcohols, amides, barbituric acid derivatives, benzodiazepine derivatives, bromides, carbamates, chloral derivatives, quinazolone derivatives and piperidinediones. Specific examples include acecarbromal, acetal, acetophenone, aldol, allobarbital, ammonium valerate, amobarbital, aprobarbital, apronalide, barbital, brallobarbital, bromisovalum, bromoform, brotizolam, butabarbital, butalbital, butallylonal, butethal, butoctamide, calcium bromolactobionate, capuride, carbocloral, carbromal, carbubarb, carfimate, chloral betaine, chloral formamide, chloral hydrate, α-chloralose, chlorhexadol, cinolazepam, clomethiazole, cyclobarbital, cyclopentobarbital, cypripedium, dexmedetomidine, dichloralphenazone, diethylbromoacetamide, doxefazepam, doxylamine, ectylurea, enallylpropymal, estazolam, etaqualone, ethchlorvynol, ethinamate, etodroxizine, etomidate, febarbamate, flunitrazepam, flurazepam, glutethimide, haloxazolam, heptabarbital, hexapropymate, hexethal, hexobarbital, hydrobromic acid, isovaleryl diethylamide, loprazolam, lormetazepam, mecloqualone, menthyl valerate, meparfynol, mephobarbital, methaqualone, methitural, methyprylon, midazolam, narcobarbital, nealbarbital, niaprazine, nimetazepam, nitrazepam, opium, paraldehyde, pentaerythritol chloral, pentobarbital, tert-pentyl alcohol, perlapine, phenallymal, phenobarbital, phenylmethylbarbituric acid, piperidione, propallylonal, propiomazine, proxibarbal, pyrithyldione, quazepam, reposal, rilmazafone, secobarbital, sulfonethylmethane, sulfonmethane, talbutal, temazepam, tetrabarbital, thalidomide, triazolam, 2,2,2-trichloroethanol, triclofos, trimetozine, valdetamide, vinbarbital, vinylbital, zaleplon, zolpidem, zopiclone, pharmaceutically acceptable salts thereof, and combinations thereof.

Nonlimiting examples of tranquilizers that can be useful in the further active agent include without limitation anxiolytics such as arylpiperazines, benzodiazepine derivatives and carbamates. Specific examples include abecarnil, alpidem, alprazolam, benzoctamine, bromazepam, buspirone, camazepam, captodiamine, chlordiazepoxide, chlormezanone, clobazam, clorazepic acid, clotiazepam, cloxazolam, diazepam, emylcamate, enciprazine, ethyl loflazepate, etifoxine, etizolam, flesinoxan, fludiazepam, fluoresone, flutazolam, flutoprazepam, glutamic acid, halazepam, hydroxyphenamate, hydroxyzine, ipsapirone, ketazolam, lesopitron, lorazepam, loxapine, medazepam, meprobamate, metaclazepam, mexazolam, nordazepam, oxazepam, oxazolam, pazinaclone, pinazepam, prazepam, suriclone, tandospirone, tofisopam, tybamate, valnoctamide, pharmaceutically acceptable salts thereof, and combinations thereof.

Nonlimiting examples of antidepressants that can be useful in the further active agent include without limitation bicyclic, tricyclic and tetracyclic antidepressants, hydrazides, hydrazines, phenyloxazolidinones and pyrrolidones. Specific examples include adinazolam, adrafinil, amineptine, amitriptyline, amitriptylinoxide, amoxapine, befloxatone, bupropion, butacetin, butriptyline, caroxazone, citalopram, clomipramine, cotinine, demexiptiline, desipramine, dibenzepin, dimetacrine, dimethazan, dioxadrol, dothiepin, doxepin, duloxetine, etoperidone, femoxetine, fencamine, fenpentadiol, fluacizine, fluoxetine, fluvoxamine, hematoporphyrin, hypericin, imipramine, imipramine N-oxide, indalpine, indeloxazine, iprindole, iproclozide, iproniazid, isocarboxazid, levophacetoperane, lofepramine, maprotiline, medifoxamine, melitracen, metapramine, metralindole, mianserin, milnacipran, minaprine, mirtazapine, moclobemide, nefazodone, nefopam, nialamide, nomifensine, nortriptyline, noxiptilin, octamoxin, opipramol, oxaflozane, oxitriptan, oxypertine, paroxetine, phenelzine, piberaline, pizotyline, prolintane, propizepine, protriptyline, pyrisuccideanol, quinupramine, reboxetine, ritanserin, roxindole, rubidium chloride, sertraline, sulpiride, tandospirone, thiazesim, thozalinone, tianeptine, tofenacin, toloxatone, tranylcypromine, trazodone, trimipramine, tryptophan, venlafaxine, viloxazine, zimeldine, pharmaceutically acceptable salts thereof, and combinations thereof.

Nonlimiting examples of neuroprotective agents that can be useful in the further active agent include aptiganel, citicoline, dexanabinol, ebselen, licostinel, lubeluzole, remacemide, repinotan, riluzole, xaliproden, ziconotide, pharmaceutically acceptable salts thereof, and combinations thereof.

In a particular embodiment, a compound of Formula (I), for example rotigotine, is administered in combination with dextromethorphan.

Combination therapy can involve, for example, simultaneous or sequential delivery of the two active agents. Sequential administration can be achieved using a single dosage form, for example a dosage form such as an oral tablet that has two layers with different release profiles for the two active ingredients. One of ordinary skill in the art will appreciate that various other forms of administration and application patterns are conceivable within the context of the present disclosure, all of which form subject matter of the invention.

Rotigotine and other 2-aminotetralin compounds of Formula (I) are structurally different, as illustrated below, from dopamine agonists such as pramipexole and ropinirole previously reported to be useful for treatment of pain.

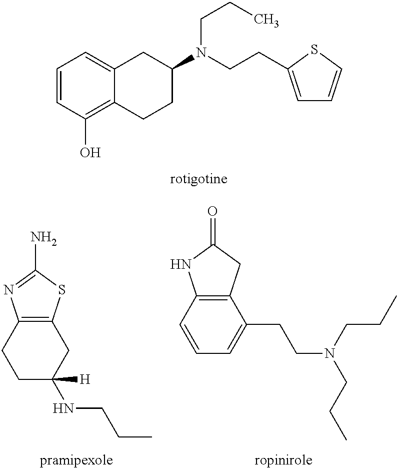

Rotigotine also differs from pramipexole and ropinirole in its receptor affinity profile. Rotigotine is a non-ergolinic dopamine agonist binding to all dopamine receptors, with a clear preference for the D3 receptor. It has greater affinity for the D1 receptor than pramipexole and ropinirole and is also an agonist of the 5-HT_{1A} receptor and an antagonist of the α2B receptor. It is believed, without being bound by theory, that the affinity of rotigotine for the 5-HT_{1A} receptor is of particular significance, as dysfunction in serotonin (5-HT) and norepinephrine (NE) transmission may influence pain in patients with fibromyalgia. See, for example, Littlejohn & Guymer, Current Pharmaceutical Design 12:3-9 (2006).

Compounds of Formula (I), for example rotigotine, may provide a lower likelihood of augmentation and rebound effects in comparison to other dopaminergic agents, such as for example levodopa. In a recent restless legs syndrome (RLS) study, a number of patients receiving long-term treatment with pramipexole experienced augmentation effects. See Happe et al., CNS Drugs 18(1):27-36 (2004). Augmentation effects include intensification of symptoms following long-term use of a compound. Rebound effects include increased occurrence of symptoms as the compound dosage wears off.

As shown in the following examples, rotigotine, an illustrative substituted 2-aminotetralin compound of Formula (I), has analgesic properties and shows dose-dependent antinociceptive effect in an animal model of non-inflammatory musculoskeletal pain.

### EXAMPLES

### Example 1

### Formalin Pain Model

The mouse formalin test is a chemically-induced sustained pain model with biphasic changes of nociceptive behavior. In mice, the test measures duration of hind paw licking following subplantar injection of formalin. Formalin produces a characteristic biphasic pain response. The early phase reflects acute pain and the late phase the chronic pain in which spinal/supraspinal plasticity of nociception is considered as a molecular basis. These features have resulted in the formalin test being accepted as a valid model of persistent clinical pain such as neuropathic, nociceptive and inflammatory pain. See, for example, Hunskaar et al., J. Neuroscience Meth. 14:69-76 (1985).

Rotigotine (SPM-962 base) was evaluated for possible analgesic activity in the mouse formalin test in which hind paw licking time was measured at 5-minute intervals for 30 minutes following subplantar injection of formalin.

Rotigotine was administered intraperitoneally to 10 CD-1 (Crl.) derived mice weighing 22±2 g (provided by BioLasco Taiwan under Charles River Laboratories Technology License). Rotigotine (3, 1 and 0.3 mg/kg) in a vehicle (5 ml/kg) comprising 0.2% HPMC (hydroxypropylmethylcellulose) and 0.9% NaCl, and vehicle (5 ml/kg) alone as a control, were each administered by intraperitoneal (i.p.) injection 30 minutes before subplantar injection of formalin (0.02 ml, 2% solution). Reduction of formalin-induced hind paw licking time was recorded at 5-minute intervals during the 0 to 30 minute period after formalin injection. A reduction of licking time of ≧50% indicates significant analgesic and anti-phlogistic activity. Statistical analysis was performed using one-way ANOVA (analysis of variance) and Dunnett's test to compare rotigotine-treated and vehicle control groups. Observation of animals for acute toxic symptoms and autonomic effects was performed before formalin injection.

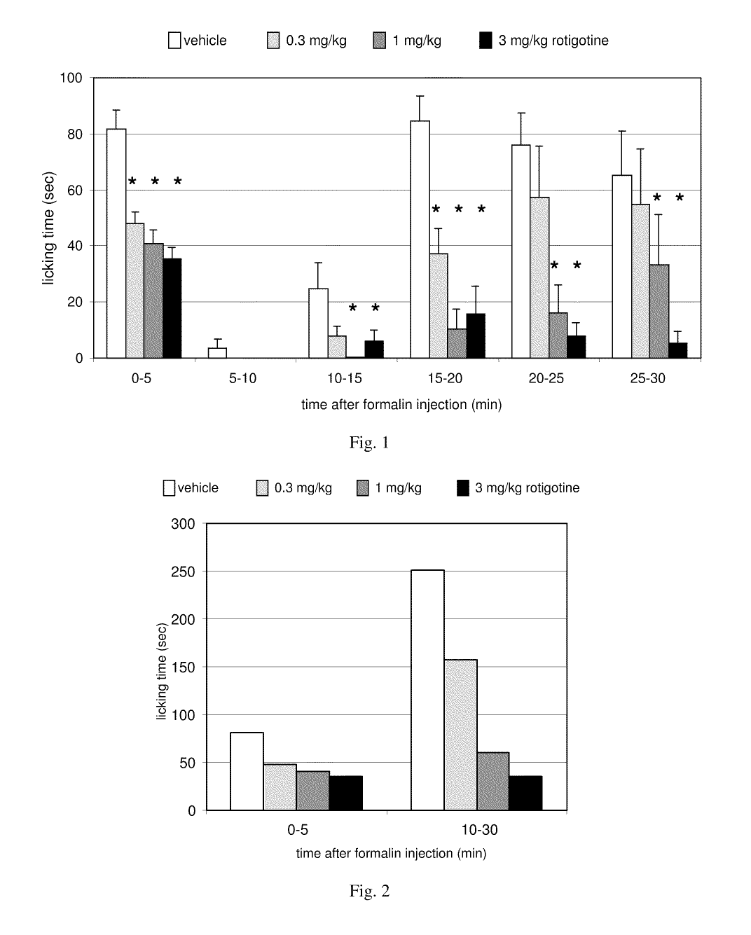

Results are summarized in Table 1, and shown graphically in FIGS. 1 and 2. Rotigotine exhibited significant dose-dependent analgesic activity in early and late phase. Significant reduction in formalin-induced hind paw licking time was observed over the vehicle control with rotigotine at all three doses at least at the 0-5 and 15-20 minute intervals. Significant reduction in hind paw licking time was observed with the 1 mg/kg and 3 mg/kg rotigotine-treated groups at the 10-15, 20-25 and 25-30 minute intervals. No significant central or autonomic signs were observed.

TABLE 1
Results of the mouse formalin test

| | Hind paw licking time (seconds) | | | | | | |
|---|---|---|---|---|---|---|---|
| Treatment | Time (min) | 0-5 | 5-10 | 10-15 | 15-20 | 20-25 | 25-30 |
| Rotigotine 3 mg/kg | Average | 35.6 | 0 | 6 | 15.9 | 7.8 | 5.5 |
| | SEM | 3.9 | 0 | 4.1 | 10 | 4.8 | 4.3 |
| | % Inhibition | (56) | (100) | (76) | (81) | (90) | (92) |
| Rotigotine 1 mg/kg | Average | 41.0 | 0 | 0.2 | 10.3 | 16.3 | 33.5 |
| | SEM | 4.6 | 0 | 0.2 | 7.1 | 9.9 | 17.9 |
| | % Inhibition | (50) | (100) | (99) | (88) | (79) | (49) |
| Rotigotine 0.3 mg/kg | Average | 47.9 | 0.0 | 8.0 | 37.3 | 57.2 | 54.8 |
| | SEM | 4.1 | 0.0 | 3.3 | 8.9 | 18.6 | 19.8 |
| | % Inhibition | (41) | (100) | (67) | (56) | (25) | (16) |
| Vehicle | Average | 81.6 | 3.7 | 24.6 | 84.5 | 76.1 | 65.4 |
| | SEM | 6.9 | 3.2 | 9.4 | 9.1 | 11.3 | 15.7 |

SEM = standard error of the mean

### Example 2

### TNF Model of Muscular Mechanical Hyperalgesia

The TNF test is used as a model of muscular mechanical hyperalgesia, which occurs in human fibromyalgia, myofascial pain or back pain.

Intramuscular injection of tumor necrosis factor alpha (TNF) induces mechanical muscle hyperalgesia in rats. This is quantified by measuring the withdrawal threshold to muscle pressure and the grip strength. Mechanical withdrawal threshold to muscle pressure is measured with an analgesimeter exerting pressure on the gastrocnemius muscle previously injected with TNF. Forelimb grip strength is measured with a digital grip force meter after TNF injection into biceps brachii muscles. TNF injections do not lead to morphological damage of the muscle. See, for example, Schafers et al., Pain 104(3):579-588 (2003).

Pain on palpation of muscles without morphological abnormalities is typical of fibromyalgia, myofascial pain or back pain in humans. Thus, the model of intramuscular injection of TNF can be used as a model of muscle pain related to fibromyalgia, myofascial pain or back pain. In this model the antinociceptive action of a test compound can be determined, by comparison with a control drug, for example a non-opioid analgesic such as metamizol or an anticonvulsant such as pregabalin or gabapentin.

### Animals, Induction of Muscle Pain

Adult male Sprague Dawley rats, from Charles River Sulzfeld, Germany, with a body weight of 220 g to 300 g were group-housed (3 animals per cage) and maintained in a room with controlled temperature (21-22° C.) and a reversed light-dark cycle (12 h/12 h) with food and water available ad libitum.

Recombinant rat TNF obtained from R&D Systems, Minneapolis, Minn. was diluted in 0.9% NaCl and used in a concentration of 1 μg in 50 μl Injections to induce muscle pain were performed on rats under a short halothane narcosis with a 30 g needle bilaterally into the gastrocnemius, or into the biceps brachii muscle. All rats were habituated to the behavioral tests before injections and baseline values were recorded over three test days.

### Behavioral Readout: Muscle Pressure (Randall-Selitto)

Mechanical withdrawal thresholds to muscle pressure were measured with an analgesimeter (Ugo Basile, Comerio, Italy) according to the Randall-Selitto method. The rats were permitted to crawl into a sock, allowing them to relax. The hind limbs of the rats were positioned such that an increasing pressure was applied to the gastrocnemius muscle (maximum 250 g). The pressure needed to elicit withdrawal was recorded. Means of 3 trials for each hind limb were calculated (interstimulus interval of >30 sec). Three pre-tests were performed on days -3, -2 and -1, testing the left and right side in succession. Pre-test values varied only minimally over these 3 days. The mean withdrawal threshold for the 3 pre-test days was determined and taken for analysis. Only animals with a significant TNF effect were included for further analysis.

The rats were injected with TNF into the gastrocnemius muscle. After 18 hours, the rats were tested for pressure hyperalgesia pre-application and 15 to 60 minutes post-application of rotigotine.

### Behavioral Readout: Grip Strength

Grip strength of the rat forelimbs was tested with a digital grip force meter (DFIS series, Chatillon, Greensboro, N.C.).

Three pre-tests were performed on days -3, -2 and -1. Since no relevant training effect for the grip strength testing could be observed, the baseline was calculated as mean of the 3 pre-test measurements and taken for further analysis. The effect of TNF injection was calculated for each animal separately and only animals with a significant TNF effect were included for further analysis.

Rats were injected with TNF into the biceps brachii muscle. Six hours later, grip strength of the forelimbs was tested with a digital grip force meter. The rats were positioned to grab a grid with the forelimbs and were gently pulled so that grip strength could be recorded. Means of ten trials were calculated.

### Application Protocol

A pilot study was performed to show that injection of 1 μg TNF intramuscularly (i.m.) into the gastrocnemius muscle was sufficient to induce pressure hyperalgesia. The rats were then placed in groups of 10 and treated with 0.3, 1.0 or 3.0 mg/kg rotigotine or vehicle i.p. Injection volume of i.p. injections was 0.5 ml/kg (weight dependent). Grip strength was again tested after 15 to 60 minutes, following injection of the rotigotine.

### Data Presentation and Statistics

Data are shown in graphs displaying means and SEMs. Pre- and post-treatment data were compared by ANOVA (analysis of variance) and a Tukey post hoc test. Means of treatment groups were compared using a one-way ANOVA and Bonferroni's post hoc test, or a Mann-Whitney-U test for comparison of metamizol versus vehicle treatment groups. Maximal possible effects (MPE) were calculated for all types of treatment. Only rats in which withdrawal thresholds were significantly reduced after TNF injection were included.

### Results

Withdrawal thresholds to pressure applied percutaneously to muscle were markedly reduced after TNF injection in most rats.

This primary muscular hyperalgesia parallels tenderness to palpation that is observed clinically in patients with myalgia, such as myofascial pain, fibromyalgia and back pain. See McCain in Wall & Melzack, eds., Textbook of Pain. New York, N.Y.: Churchill Livingstone (1994), pp. 475-493.

Tenderness to palpation is a primary criterion for diagnosis of muscle pain under clinical and experimental human conditions. See the publications individually cited below.

Wolfe et al., Arthritis Rheum. 33:160-172 (1990).

Arendt-Nielsen, Proc. 8th World Congr. Pain pp. 393-425, IASP Press, Seattle (1997).

Table 2 shows the absolute values of withdrawal thresholds to pressure without injection of TNF. Withdrawal thresholds remained stable after phosphate-buffered saline (PBS) injection. Significantly higher withdrawal thresholds were seen with rotigotine 1 mg/kg.

TABLE 2
Effect of rotigotine on withdrawal pressure (without TNF)

| Group | Mean (g) | SEM |
|---|---|---|
| control | 9.1; 8.6; 9.6 | 0.3; 0.3; 0.3 |
| saline | 9.2; 9.3; 9.7 | 0.3; 0.3; 0.5 |
| rotigotine, 0.3 mg/kg | 9.1 | 0.4 |
| rotigotine, 1 mg/kg | 10.8* | 0.6 |
| rotigotine, 3 mg/kg | 9.7 | 0.4 |

SEM = standard error of the mean;
*P < 0.05

Table 3 shows the absolute values of withdrawal thresholds to pressure with injection of TNF.

TABLE 3
Effect of rotigotine on withdrawal pressure (with TNF)

| Group | Mean (g) | SEM |
|---|---|---|
| control | 9.8; 9.6; 9.7; 9.6 | 0.3; 0.3; 0.2; 0.2 |
| TNF | 5.7; 6.0; 6.1; 6.8 | 0.2; 0.2; 0.2; 0.2 |
| PBS | 6.4 | 0.2 |
| rotigotine, 0.3 mg/kg | 5.6 | 0.2 |
| rotigotine, 1 mg/kg | 6.5 | 0.2 |
| rotigotine, 3 mg/kg | 6.5 | 0.3 |

SEM = standard error of the mean;
* P < 0.05

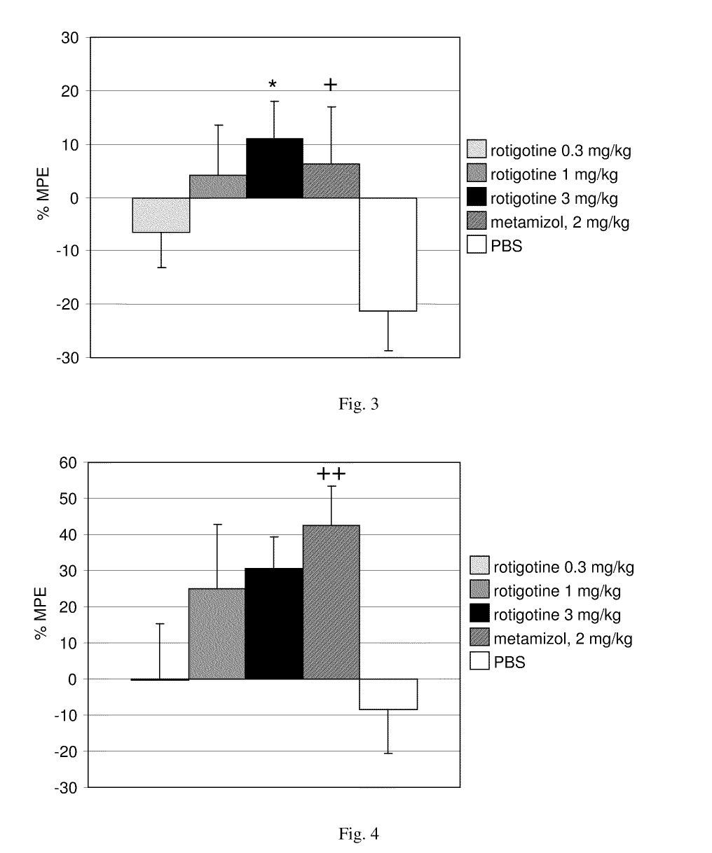

As shown in Table 4 and FIG. 3, the percent of maximal possible effect (% MPE) was significantly different from vehicle for rotigotine 3 mg/kg and metamizol 2 mg/kg. Vehicle (PBS) had no effect.

TABLE 4
% MPE of rotigotine and metamizol on withdrawal pressure

| Group | Mean (%) | SEM |
|---|---|---|
| rotigotine, 0.3 mg/kg | −6.6 | 6.6 |
| rotigotine, 1 mg/kg | 4.8 | 9.4 |
| rotigotine, 3 mg/kg | 10.9 (*) | 7.1 |
| metamizol, 2 mg/kg | 6.2 (+) | 10.7 |
| PBS | −21.3 | 7.5 |

SEM = standard error of the mean
(*) P < 0.05 (ANOVA + Bonferroni post hoc) versus PBS
(+) P < 0.05 (Mann-Whiney-U test) versus PBS

Table 5 shows the absolute values of grip strength without injection of TNF. The grip strength values were all stable after saline injection.

TABLE 5
Effect of rotigotine on grip strength (without TNF)

| Group | Mean (N) | SEM |
|---|---|---|
| control | 8.5; 8.3; 9.1 | 0.2; 0.2; 0.1 |
| saline | 8.1; 8.1; 9.3 | 0.7; 0.5; 0.3 |
| rotigotine, 0.3 mg/kg | 7.5 | 0.5 |
| rotigotine, 1 mg/kg | 7.8 | 0.3 |
| rotigotine, 3 mg/kg | 9.0 | 0.1 |

SEM = standard error of the mean

Table 6 shows the absolute values of grip strength after injection of TNF.

TABLE 6
Effect of rotigotine on grip strength (with TNF)

| Group | Mean (N) | SEM |
|---|---|---|
| control | 9.1; 9.1; 8.9; 9.6 | 0.2; 0.1; 0.1; 0.1 |
| TNF | 7.7; 7.0; 7.0; 7.1 | 0.2; 0.4; 0.2; 0.3 |
| PBS | 7.1 | 0.2 |
| rotigotine, 0.3 mg/kg | 7.8 | 0.3 |
| rotigotine, 1 mg/kg | 7.6 | 0.2 |
| rotigotine, 3 mg/kg | 7.6 | 0.3 |

SEM = standard error of the mean

As shown in Table 7 and FIG. 4, the percent of maximal possible effect (% MPE) was significantly different from vehicle for metamizol 2 mg/kg. Vehicle (PBS) had no effect.

TABLE 7
% MPE of rotigotine and metamizol on grip strength

| Group | Mean (%) | SEM |
|---|---|---|
| rotigotine, 0.3 mg/kg | −0.2 | 15.5 |
| rotigotine, 1 mg/kg | 24.9 | 17.9 |
| rotigotine, 3 mg/kg | 30.5 | 8.9 |
| metamizol, 2 mg/kg | 42.4 (++) | 11.1 |
| PBS | −8.4 | 12.2 |

SEM = standard error of the mean
(++) P < 0.01 (Mann-Whiney-U test) versus PBS

From the results of this study it can be concluded that rotigotine induces a dose-dependent reduction of muscular hyperalgesia induced by TNF injected into muscle.

### Example 3

### Parallel, Randomized, Double-blinded, Placebo-Controlled Proof of Concept Trial to Assess the Efficacy and Safety of Rotigotine in Subjects with Signs and Symptoms Associated with Fibromyalgia Syndrome

This proof of concept trial investigates the efficacy and safety of 2 doses of rotigotine in adult male and female subjects with fibromyalgia syndrome. This trial is a randomized, double-blind, placebo-controlled, multicenter trial.

The overall post-baseline duration of treatment is 13 weeks. The trial consists of a 4-week Titration Phase, an 8-week Maintenance Phase, a 1-week De-escalation Phase, and a 2-week Safety Follow-Up Phase. If subjects meet the eligibility criteria, they are randomized to receive either rotigotine 4 mg/24 hr, rotigotine 8 mg/24 hr, or placebo during the Maintenance Phase. Subjects assigned to rotigotine are titrated at weekly intervals of 2 mg/24 hr until they reach 4 mg/24 hr or 8 mg/24 hr. All subjects completing the 4-week Titration Phase enter an 8-week Maintenance Phase. No dose adjustment is allowed during the Maintenance Phase. The Treatment Phase is defined as the combined Titration and Maintenance Phases.

The primary variable for this trial is the change in average Likert pain score from baseline to the last 2 weeks of the Treatment Phase. The secondary efficacy variables are the Fibromyalgia Impact Questionnaire (FIQ) total score and associated subscores, the total myalgic score (the numerical assessment of pain from palpation of 18 possible tender points), subject's perception of interference with sleep and general activity, and the Patient Global Impression of Change (PGIC) scale. Other variables include the Beck Depression Inventory-II (BDI-II), Hospital Anxiety and Depression Scale (HADS) depression and anxiety subscale scores, use of rescue medication (including alcohol) for pain, fibromyalgia symptom checklist, presence of impulse control disorders, sleep attacks, menstrual/sexual function, and pharmacokinetic assessments. Subjects use a paper diary in the morning and evening to record pain intensity, pain interference with sleep and general activity, and use of rescue medication.

Approximately 25 sites are selected to meet the recruitment timeline. In order to randomize 240 subjects (80 subjects per treatment arm) approximately 480 subjects are enrolled.

### Trial Design

Variables to be assessed are the following.

Primary variable: Within-subject change in average daily pain score from baseline to the last 2 weeks of the Treatment Phase using an 11-point Likert scale (0-10).

Secondary variables (efficacy): Within-subject change from baseline to endpoint in FIQ (0-100); within-subject change from baseline to endpoint in total myalgic score (0-54); within-subject change in average daily interference with sleep from baseline to the last 2 weeks of the Treatment Phase using an 11-point Likert scale (0-10); within-subject change in average daily interference with general activity from baseline to the last 2 weeks of the Treatment Phase using an 11-point Likert scale (0-10); global perception of change in pain from baseline to endpoint using the PGIC scale; within-subject change from baseline to the last 2 weeks of the Treatment Phase in average morning pain score; within-subject change from baseline to the last 2 weeks of the Treatment Phase in average evening pain score.

Secondary variables (other): Within-subject change from baseline on the BDI-II; within-subject change from baseline on the HADS depression and anxiety subscale scores; use of rescue medication (including alcohol) for pain; changes in fibromyalgia symptom checklist; presence of impulse control disorders (assessed by the Jay Modified Minnesota Impulsive Disorders Interview (MIDI)); plasma concentrations of rotigotine.

Secondary variables (safety): Observation and assessment of adverse events (AEs); changes in laboratory parameters (including endocrine parameters); changes in vital sign measurements (blood pressure, pulse, temperature, body weight); changes in physical examination findings; changes in 12-lead electrocardiograms (ECGs); presence of sleep attacks; changes in menstrual/sexual function; subject withdrawals due to AEs.

### Trial Description

The overall trial consists of the Screening Phase through the end of the Safety Follow-Up Phase (see Table 8).

TABLE 8
Trial description

| | | |
|---|---|---|
| Screening (Visit 1) | Washout of prohibited | up to 4 weeks |
| | medications | |
| | Baseline Diary Phase | 7 days prior to Baseline |
| | | (Visit 2)* |
| Baseline | | |
| (randomization; | | |
| Visit 2) | | |
| Titration | | 4 weeks |
| Maintenance | | 8 weeks |
| De-escalation | | 1 week |
| Safety Follow-Up | | 2 weeks |

*Subjects complete the diary each day beginning at Screening (Visit 1); the 7 days prior to Baseline (Visit 2) are used to determine eligibility for randomization.

### Trial Treatment

Subjects who complete the Screening Phase enter the Titration Phase at Visit 2 (Baseline) and are randomized to 1 of 3 different treatment groups: Rotigotine 4 mg/24 hr, Rotigotine 8 mg/24 hr and Placebo.

Two different patch sizes are used (10 cm² and 20 cm²). Active patches deliver either 2 mg/24 hr or 4 mg/24 hr of rotigotine. Placebo patches are matched according to size and appearance.

### Methods for Assessing Efficacy Parameters

The efficacy parameters are assessed using—among others—the following rating scales, questionnaires, and assessments.

Likert scales: For the subject's assessment of his/her condition, an 11-point Likert scale is used. Subjects complete the diary daily in the morning and evening as specified. Pain scale—the subject rates his/her average pain over the last 12 hours, from 0 (no pain) to 10 (worst pain ever experienced) (morning and evening diary). Sleep scale—the subject rates quality of sleep, from 0 (very good sleep) to 10 (very poor sleep) (morning diary only), if sleep was sufficient (Yes/No), and if the subject awoke rested (Yes/No). General activity scale—the subject rates how the pain has interfered with general activity over the past 12 hours, from 0 (did not interfere) to 10 (completely interfered) (evening diary only).

Fibromyalgia Impact Questionnaire (FIQ): The FIQ is a self-administered instrument composed of 20 questions. It is completed at the beginning of the visit. The first item contains 11 questions related to physical functioning; each question is rated on a 4-point Likert-type scale. Questions 12 and 13 ask the subject to mark the number of days he/she felt well and the number of days he/she was unable to work (including housework) because of fibromyalgia symptoms. Questions 14 through 20 are horizontal linear scales marked in 10 increments on which the subject rates work difficulty, pain, fatigue, morning tiredness, stiffness, anxiety, and depression.

Patient Global Impression of Change (PGIC): The PGIC is a 7-point self-administered categorical rating scale in which the subject rates the change in his/her pain since starting trial medication (from much worse (score of 1) to much better (score of 7)).

Total myalgic score: The total myalgic score is based on clinician assessment of the 18 tender points associated with fibromyalgia. The investigator should press on each tender point with enough pressure (4 kg/cm²) to have the skin under the thumbnail blanch. Each point is rated on a scale of 0 to 3 (0=no pain, 1=pain is reproduced, 2=focal response to pain, 3=subject flinches or withdraws), and the total score is summed. The maximum myalgic score is 54. Every attempt should be made to have the same clinician perform this assessment for all subjects throughout the trial.

What is claimed is:

1. A method for treating pain in a subject, comprising administering to the subject a therapeutically effective amount of rotigotine or a pharmaceutically acceptable salt, prodrug or metabolite thereof.

2. The method of claim 1, wherein the pain comprises musculoskeletal pain, fibromyalgia, myofascial pain, pain during menstruation, pain during osteoarthritis, pain during rheumatoid arthritis, pain during gastrointestinal inflammation, pain during inflammation of the heart muscle, pain during multiple sclerosis, pain during neuritis, pain during AIDS, pain during chemotherapy, tumor pain, headache, CPS, central pain, neuropathic pain, trigeminal neuralgia, shingles, stamp pain, phantom limb pain, temporomandibular joint disorder, nerve injury, migraine, post-herpetic neuralgia, neuropathic pain encountered as a consequence of injuries, amputation infections, metabolic disorders or degenerative diseases of the nervous system, neuropathic pain associated with diabetes, pseudesthesia, hypothyroidism, uremia, vitamin deficiencies or alcoholism, acute pain after injuries, postoperative pain, pain during acute gout, or pain from operations.

3. The method of claim 1, wherein the pain is musculoskeletal pain.

4. The method of claim 3, wherein the musculoskeletal pain is non-inflammatory.

5. The method of claim 3, wherein the musculoskeletal pain comprises myofascial pain or back pain.

6. The method of claim 3, wherein the subject has myofascial pain syndrome.

7. The method of claim 3, wherein muscular hyperalgesia and/or muscular allodynia are reduced.

8. The method of claim 1, wherein rotigotine hydrochloride is administered.

9. The method of claim 1, wherein rotigotine is administered parenterally, transdermally or transmucosally.

10. The method of claim 9, wherein rotigotine is administered transdermally in a transdermal therapeutic system (TTS).

11. The method of claim 10, wherein the TTS comprises a self-adhesive matrix layer comprising one or more amine-resistant silicone adhesives, said matrix layer having rotigotine free base dispersed therein in an amount of about 0.05 to about 2.5 mg/cm².

12. The method of claim 11, wherein the matrix layer of the TTS further comprises a compatibilizing agent in an amount of about 1.5% to about 5% by weight of the matrix layer, said compatibilizing agent comprising povidone, a vinylpyrrolidone/vinyl acetate copolymer and/or an ethylene/vinyl acetate copolymer.

13. The method of claim 11, wherein the matrix layer of the TTS comprises at least two amine-resistant silicone adhesives, including at least one high-tack and at least one medium-tack adhesive.

14. The method of claim 11, wherein the TTS comprises about 0.4 to about 0.5 mg/cm² rotigotine free base.

15. The method of claim 14, wherein the TTS comprises one to a plurality of patches, and wherein the TTS has a total surface area for release of the rotigotine of about 2 to about 60 cm².

16. The method of claim 15, wherein the total surface area is about 4.5, about 9, about 13.5 or about 18 cm².

17. The method of claim 10, wherein rotigotine is administered in an applied dose of about 0.05 to about 50 mg/day.

18. The method of claim 10, wherein rotigotine is administered in an applied dose of about 4 to about 20 mg/day.

19. The method of claim 10, wherein successive applications of the TTS are made to different areas of skin of the subject.

20. The method of claim 8, further comprising administering at least one further active agent.

21. The method of claim 20, wherein the at least one further active agent comprises an opioid, a CGRP antagonist, an NMDA receptor blocker, a cannabinoid, a bradykinin antagonist, acetaminophen, an NSAID, a COX-2 selective inhibitor, a sedative, an antidepressant, a tranquilizer and/or a neuroprotective agent.

22. The method of claim 20, wherein the at least one further active agent comprises dextromethorphan.

23. The method of claim 10, wherein the TTS is (a) a reference TTS having a matrix layer that consists essentially of 4.5 mg rotigotine free base, 1.0 mg povidone, at least one high-tack and at least one medium-tack silicone adhesive, 0.01 mg ascorbyl palmitate, 0.025 mg DL-α-tocopherol and 0.00045 mg sodium metabisulfite per 10 cm², and having a total surface area for release of rotigotine of about 10 to about 40 cm², or (b) a rotigotine-containing TTS that is substantially bioequivalent to said reference TTS.

24. The method of claim 23, wherein the pain is non-inflammatory musculoskeletal pain.

25. The method of claim 24, wherein the musculoskeletal pain comprises myofascial pain or back pain.

26. The method of claim 1, wherein the pain is neuropathic pain encountered as a consequence of a metabolic disorder or degenerative disease of the nervous system.
